(19) United States
(12) Patent Application Publication
(10) Pub. No.: US 2014/0222127 A1
(43) Pub. Date: Aug. 7, 2014

(54) HELICAL VASCULAR REINFORCEMENT DEVICE

(71) Applicant: Empire Technology Development LLC, Wilmington, DE (US)

(72) Inventors: John Mathew Adams, Snohomish, WA (US); Daniel Hawkins, Bellevue, WA (US)

(73) Assignee: Empire Technology Development LLC, Wilmington, DE (US)

(21) Appl. No.: 14/116,120

(22) PCT Filed: Dec. 18, 2012
(86) PCT No.: PCT/US12/70425

§ 371 Date: Nov. 6, 2013

(51) Int. Cl. A61F 2/92; A61L 27/58; A61F 2/966

(52) U.S. Cl. CPC A61F 2/92; A61F 2/966; A61L 27/58
USPC 623/1.11; 623/1.22

(57) ABSTRACT

Technologies are generally provided for a vascular reinforcement device for preventing compression of a blood vessel in the presence of forces applied externally to the blood vessel which may occur due to pregnancy and obesity. The vascular reinforcement device may include a coil which may be wrapped around a left renal vein at a position where the left renal vein crosses the aorta for preventing compression of the left renal vein against the rigid aorta. The coil may be uncoiled into a long a flat position for percutaneous delivery to the abdominal area employing a laparoscopic procedure. The coil may be inserted into a delivery tube in the uncoiled position and delivered through the delivery tube employing a guiding tool. As the coil emerges from the delivery tube, the coil may be configured to form into the coiled position around the left renal vein.

### BACKGROUND

Unless otherwise indicated herein, the materials described in this section are not prior art to the claims in this application and are not admitted to be prior art by inclusion in this section.

Preeclampsia is a pregnancy induced hypertension that can be associated with proteinuria (an excess of serum proteins in the urine) and edema. Preeclampsia typically occurs in 5-10% of pregnancies, and is characterized by symptoms such as swelling, sudden weight gain, headaches and changes in vision. Preeclampsia can progress to eclampsia, with cerebral symptoms leading to convulsions. The condition is associated with systemic vasospasm wherein arteries throughout the body narrow. This can lead to multi-organ system dysfunction wherein many organs of the body, including the kidneys, brain, eyes, liver, etc., are unable to function normally because of altered blood flow and increased blood pressure. Currently the only effective treatment is delivery of the fetus and placenta. Typically, preeclampsia occurs after 20 weeks gestation (in the late 2nd or 3rd trimester), though it can occur earlier.

While the cause of preeclampsia is still being debated, inadequate blood supply to the placenta, abnormalities in the immune system and maternal endothelial cell dysfunction are suspected to be involved. A theorized cause of preeclampsia is compression of the left renal vein due to increased abdominal pressures caused by the growing uterus and abdomen during pregnancy. Abdominal organs can shift due to the growing uterus and can pin the left renal vein, which passes between the vertebra and the aorta, against the rigid aorta causing the blood pressure within the left renal vein to increase substantially. The increase in blood pressure within the left renal vein activates a biological system in the kidney which in effect causes increased system blood pressure, or hypertension. Other circumstances can also trigger the hypertension symptoms, for example, obesity can cause external forces to be exerted on the left renal vein against the aorta, leading to a higher than normal renal vein pressure resulting in hypertension and preeclampsia symptoms.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

According to some examples, the present disclosure describes a vascular reinforcement device for preventing compression of a blood vessel. The vascular reinforcement device may include a coil formed of a flexible material, the coil configured to wrap around a blood vessel for reducing an external pressure applied to the blood vessel.

According to some examples, the present disclosure also describes a method of preventing compression of a vein from external bodily tissue forces. The method may include providing a coil formed of a flexible material, and wrapping the coil in around a blood vessel for reducing an external pressure applied to the blood vessel.

According to other examples, the present disclosure also describes a system for preventing compression of a vein from external bodily tissue forces. The system may include a surgical delivery tube for providing percutaneous access to an internal area of a body via at least one incision and a vascular reinforcement device including a coil formed of a flexible material, the coil configured to wrap around a blood vessel for reducing an external pressure applied to the blood vessel, wherein the coil may be configured in an initial uncoiled position for delivery to the internal area of the body via the delivery tube and deployed into the coiled position around the blood vessel.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example anatomical layout of the abdomen including the kidneys, left renal vein, inferior vena cava, and the aorta;

FIG. 2 illustrates an arrangement of the left renal vein and renal artery and a schematic of the corresponding blood pressures;

FIG. 3 illustrates the location of the left renal vein where it crosses the aorta and a schematic diagram of the blood pressure when the left renal vein is compressed;

FIG. 4 illustrates a vascular reinforcement device for protecting the left renal vein from external pressures;

FIG. 5A illustrates a vascular reinforcement device in an uncoiled position and stored in a delivery tool;

FIG. 5B illustrates a vascular reinforcement device as it is pushed out of the delivery tool for deployment into a coiled position around the left renal vein; and

FIG. 6 illustrates a vascular reinforcement device being delivered via a delivery tool and deployed into a coiled position around the left renal vein; all arranged in accordance with at least some embodiments as described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to compositions, methods, apparatus, systems, devices, and/or computer program products related to providing a vascular reinforcement device for preventing compression of a vein.

Briefly stated, technologies are generally provided for a vascular reinforcement device for preventing compression of a blood vessel in the presence of forces applied externally to the blood vessel which may occur due to pregnancy and obesity. The vascular reinforcement device may include a coil which may be wrapped around a left renal vein at a position where the left renal vein crosses the aorta for preventing compression of the left renal vein against the rigid aorta. The coil may be uncoiled into a long a flat position for percutaneous delivery to the abdominal area employing a laparoscopic procedure. The coil may be inserted into a delivery tube in the uncoiled position and delivered through the delivery tube employing a guiding tool. As the coil emerges from the delivery tube, the coil may be configured to form into the coiled position around the left renal vein

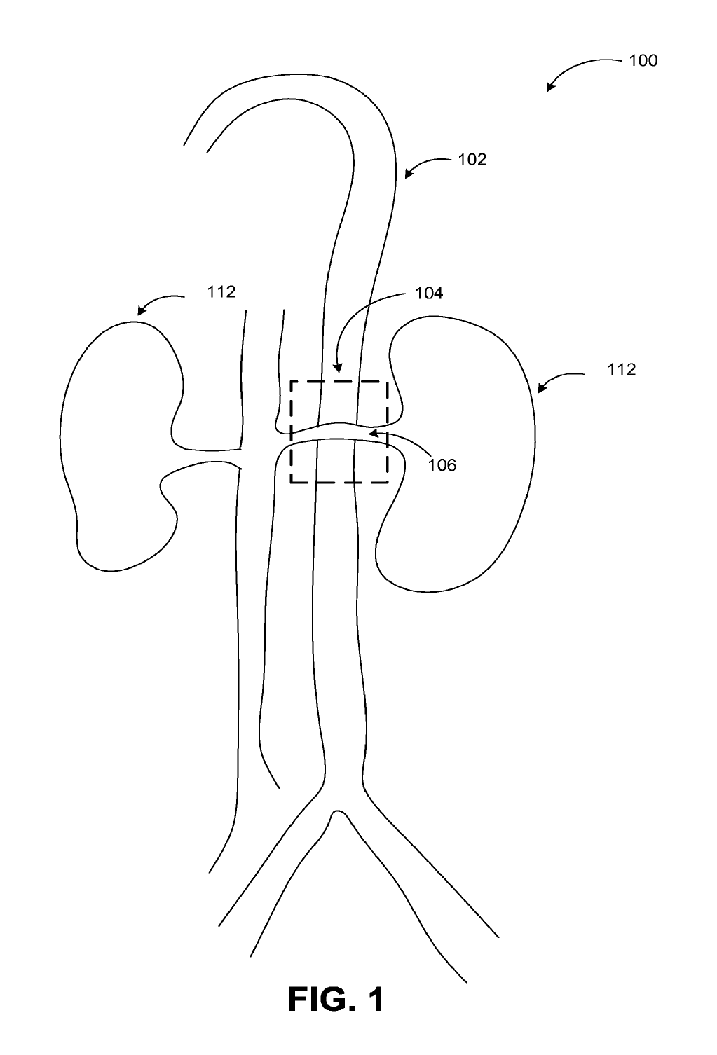

FIG. 1 illustrates an example anatomical layout of the abdomen including the kidneys, left renal vein, inferior vena cava, and the aorta, arranged in accordance with at least some embodiments as described herein. As illustrated in diagram 100, the abdominal cavity includes a right kidney 110, a left kidney 112, inferior vena cava 114, aorta 102, left renal vein 106, and right renal vein 116. The left renal vein 106 connects the left kidney 112 to the inferior vena cava 114 for support blood flow from the left kidney 112 through the inferior vena cava 114 and back to the heart. The left renal vein 106 passes over 104 and is immediately adjacent to the aorta 102. The aorta 102 is a large artery and is at high fluid/blood pressure and has a rigid structure when compared to the compliant vascular structure of a relatively low pressure in the left renal vein 106.

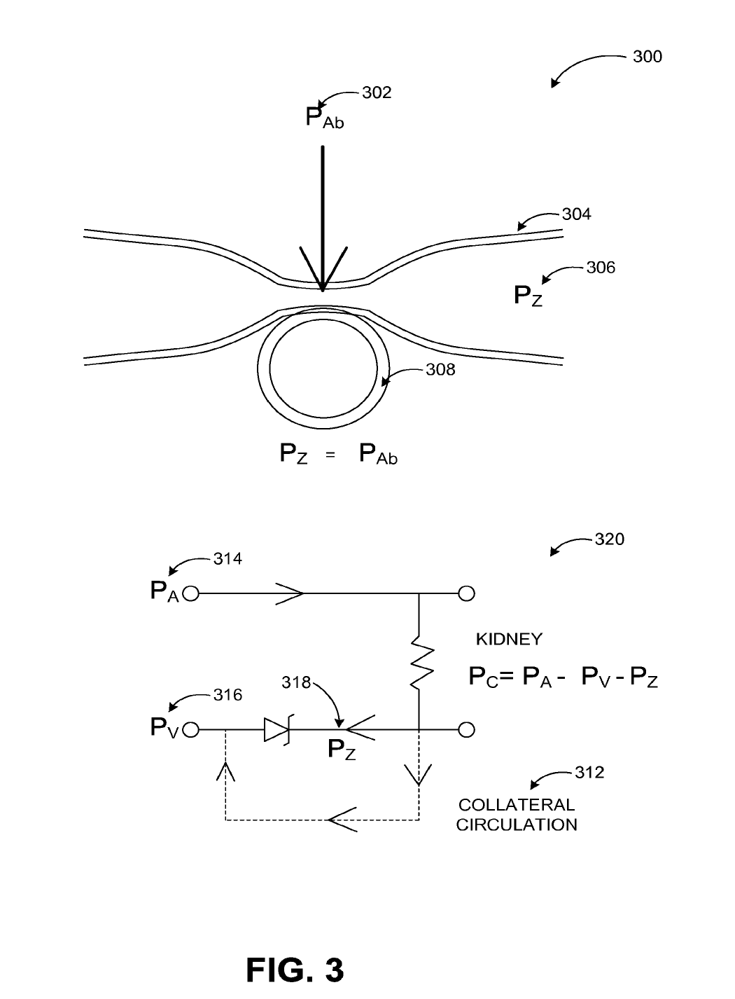

Often times the left renal vein 114 can be subjected to compressive forces within the abdominal area. For example the expansion of tissue and organs in obese persons and the expanding uterus in pregnant persons can cause compression of the left renal vein 114 against the rigid aorta 102 due to increased abdominal pressure and shifting organs. When the left renal vein 114 is compressed against the rigid aorta 102, the left renal vein 114 may distort, such that a diameter of the left renal vein 114 decreases due to the compression against the rigid aorta 102. When the diameter of the left renal vein 114 decreases, the left kidney 112 attempts to maintain a constant blood flow through the left renal vein 114 and the blood pressure within the left renal vein 114 increases on the left side of the aorta, i.e. upstream from the restriction on the left renal vein. The increased blood pressure within the left renal vein 114 is sensed as if it were a reduced aortic pressure by sensors the left kidney 112, as described further in FIG. 3, and in response, the left kidney 112 activates the renin-angiotensin system (RAS). The RAS triggers an increased production of renin enzyme which leads to increased angiotensin II production. Angiotensin II causes the blood vessels within the body to constrict, leading to systemic (whole body) vasospasm and increased systemic blood pressure, known as hypertension and, in pregnancy, as preeclampsia. Additionally, the increased systemic blood pressure leads to aldosterone production which causes water retention in the kidneys, and causes additional decreased kidney perfusion due to vasospasm, which causes the cycle of increased blood pressure regulation to continue.

To further illustrate the sensitivity of the left renal vein 114 to compressive abdominal forces, flow through a vein is equal to the pressure drop from one end of the vein to the other divided by the vascular resistance. In a vein, the vascular resistance is very sensitive to the diameter of the vein. The vascular resistance increases in inverse relation to the fourth power of radial decrease. For example, if the radius is halved, the vein pressure increases by a factor of sixteen in order to maintain constant blood flow through the vein. As a further example, if a vein is normally 6 mm in diameter and the diameter is decreased by 1 mm, the vascular pressure will double to maintain constant flow. Thus a slight compression of the left renal vein causing even a small change in diameter of the left renal vein can cause a substantial pressure increase within the vein to enable the constant flow to be maintained.

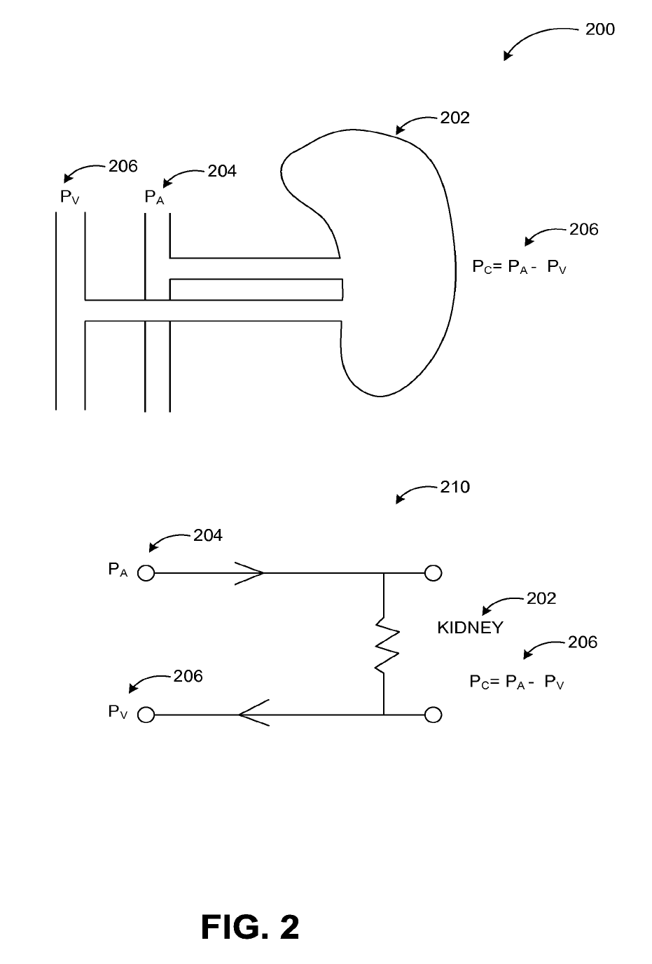

FIG. 2 illustrates an arrangement of the left renal vein and renal artery and a schematic of the corresponding blood pressures, arranged in accordance with at least some embodiments as described herein. As illustrated in diagram 200, in a typical blood pressure scenario when the left renal vein 208 is not compressed, the left kidney 202 observes the blood pressures in the aorta and the inferior vena cava. The blood pressure P_{c} 206 the left kidney 202 senses is the difference between the aortic blood pressure P_{A} 204 and the inferior vena cava blood pressure Pᵥ 206. (P_{c}=P_{A}−Pᵥ). Diagram 210 is a schematic representation of the blood pressure detected by the left kidney 202. The blood pressure P_{c} 206 the left kidney 202 sees is the difference between the aortic blood pressure P_{A} 204 and the inferior vena cava blood pressure Pᵥ 206. (P_{c}=P_{A}−Pᵥ).

FIG. 3 illustrates the location of the left renal vein where it crosses the aorta and a schematic diagram of the blood pressure when the left renal vein is compressed, arranged in accordance with at least some embodiments as described herein. As illustrated in diagram 300, if there is a pressure point 310 on the left renal vein 304 due to increased abdominal pressure P_{Ab} 302, it can compress the left renal vein 304 against the much harder aorta 308 resulting in a decreased diameter of the left renal vein 304. Pressure P_{z} is 306 the pressure needed in the left renal vein 304 to compensate for the compressive abdominal pressure P_{Ab} to allow blood to flow past the pressure point 310.

Diagram 310 illustrates a schematic layout of the pressures when there is increased abdominal pressure on the left renal vein 304. The pressure P_{c} 322 observed by the left kidney is P_{c}=P_{A}−P_{z}−Pᵥ. In this case Pc will be less than the case above where there is no compressive force on the LRV. While the aortic pressure P_{A} is the same in both models, the pressure the left kidney observes during compression of the left renal vein is less than what it should be and the kidney senses a low systematic blood pressure. When the left kidney senses a low systematic blood pressure, the left kidney begins an attempt to correct the low systematic blood pressure it senses by increasing the aortic pressure. Also illustrated in diagram 320 is an alternate collateral circulation return path for venous blood from the left kidney. This alternate or collateral circulation is common in about 85-90% of patients, and passes under the aorta. When the alternate collateral circulation is present, the present P_{z} will be zero such that the blood pressure P_{c} 322 observed by the left kidney is returned to P_{c}=P_{A}−Pᵥ, which represents the systemic blood pressure without pinching of the left renal vein due to increased abdominal pressure.

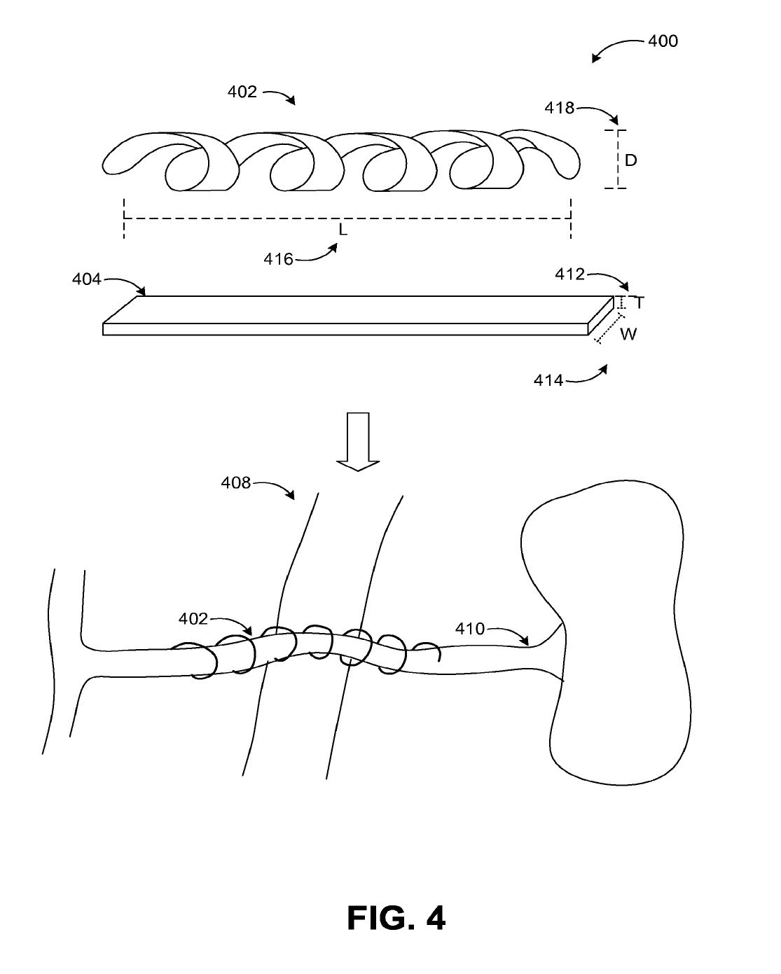

FIG. 4 illustrates a vascular reinforcement device for protecting the left renal vein from external pressures, arranged in accordance with at least some embodiments as described herein.

As illustrated in diagram 400, a coil 402 may be wrapped around the left renal vein 410 for preventing the left renal vein 410 from being compressed against the aorta 408 due to increased abdominal pressures. The coil 402 may be wrapped around the left renal vein 410 at the position where the aorta 408 crosses the left renal vein 410. The coil 402 may be composed of a flexible material, and in the uncoiled position 404, the flexible material may have a substantially rectangular cross-sectional shape. The flexible material be substantially thin, and may have a thickness (T) 412 in a range from about 1 mm to about 4 mm and a width (W) 414 in a range from about 4 mm to about 8 mm. The thin flexible material may be a polymer material such as polyamides, polyethylene, polypropylene, polyester, polyurethane, polystyrene, polysufone and/or polyethersulfone. In another embodiment, the thin flexible material for forming the coil 402 may be a metal, such as nickel titanium alloy or stainless steel. In yet another embodiment, the thin flexible material may be composed from a bio-absorbable material for enabling the coil 402 to be eventually absorbed by the body after a certain amount of time when it is no longer needed. Some example bio-absorbable materials include polyglycolic acid, polylactic acid, and polydioxanone. Some non-bioabsorbable materials include polyamides (e.g. nylon) or polypropylene, for example.

In an example embodiment, when in the coiled position, the coil 402 may have a substantially circular cross-sectional shape, and the cross-sectional internal diameter (D) 418 may be slightly larger than a diameter of the left renal vein 410, such that the coil 402 may protect the left renal vein 410 from being compressed or pinched against the aorta 408. The diameter of the left renal vein 410 can vary with each individual person, and a typical diameter of the left renal vein 410 may be in a range from about 6 mm to about 8 mm. The diameter 418 of the coil 402 in the coiled position may be in a range from about 5 mm to about 10 mm for fitting around the left renal vein 410 for protecting the left renal vein 410 from compression due to surrounding tissue and organs affected by the increased abdominal pressure. The coil 402 in the coiled position around the left renal vein 410 may be able to resist an applied external pressure within the abdomen in a range from about 50 mmHg to about 100 mmHg.

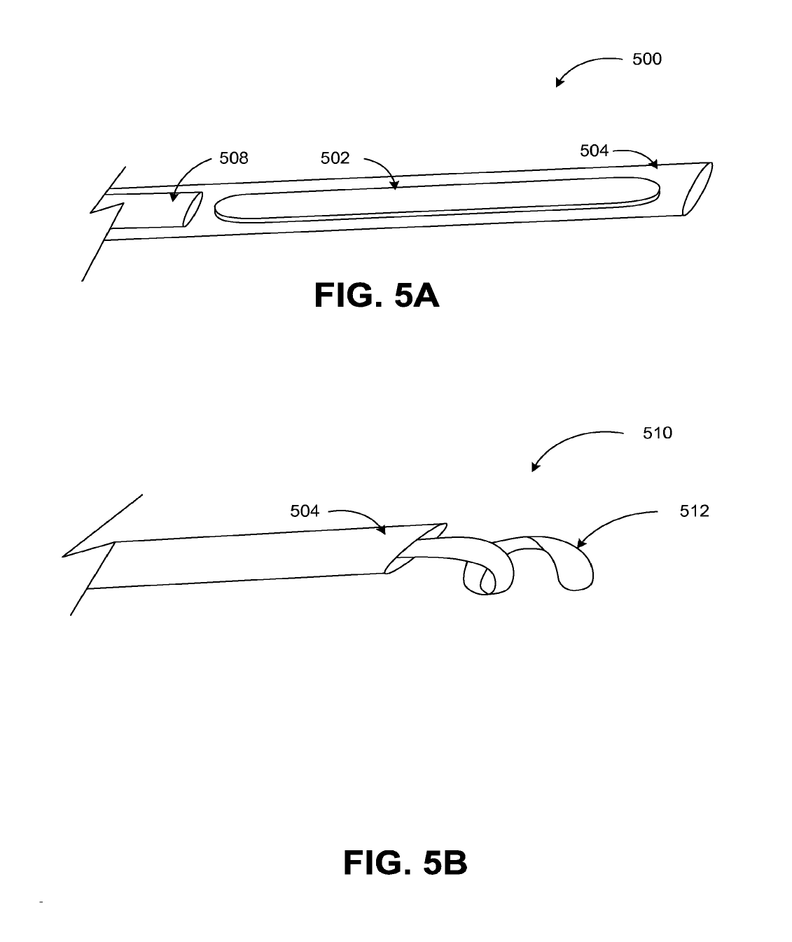

FIG. 5A illustrates a vascular reinforcement device in an uncoiled position and stored in a delivery tool, arranged in accordance with at least some embodiments as described herein. The coil 512 may be delivered and deployed into the coiled position around the left renal vein for protecting the left renal vein from compression due to increased abdominal pressure employing a laparoscopic procedure. As illustrated in diagram 500, the coil may initially be configured in an uncoiled position 502 such that the coil is in a substantially long, straight and flat form for delivery into the abdomen. The coil in the uncoiled position 502 may be inserted within a delivery tube 504 for delivering the coil into the abdominal cavity near the aorta. The coil may be guided through the delivery tube for deployment into the abdomen employing a guiding tool 508.

FIG. 5B illustrates a vascular reinforcement device as it is pushed out of the delivery tool for deployment into a coiled position around the left renal vein, arranged in accordance with at least some embodiments as described herein. As illustrated in diagram 510, the coil 512 may be delivered through the delivery tube 504 into the abdomen employing a guiding tool, and may be deployed into the coiled position over a left renal vein at a location where the left renal vein crosses the aorta. As the coil 512 emerges from the open distal end of the delivery tube 504, the coil 512 may form into the coiled position around the blood vessel. The coil 512 may be composed of a shape memory material such that the coil 512 may return to the coiled position shape when it emerges from the delivery tube 504.

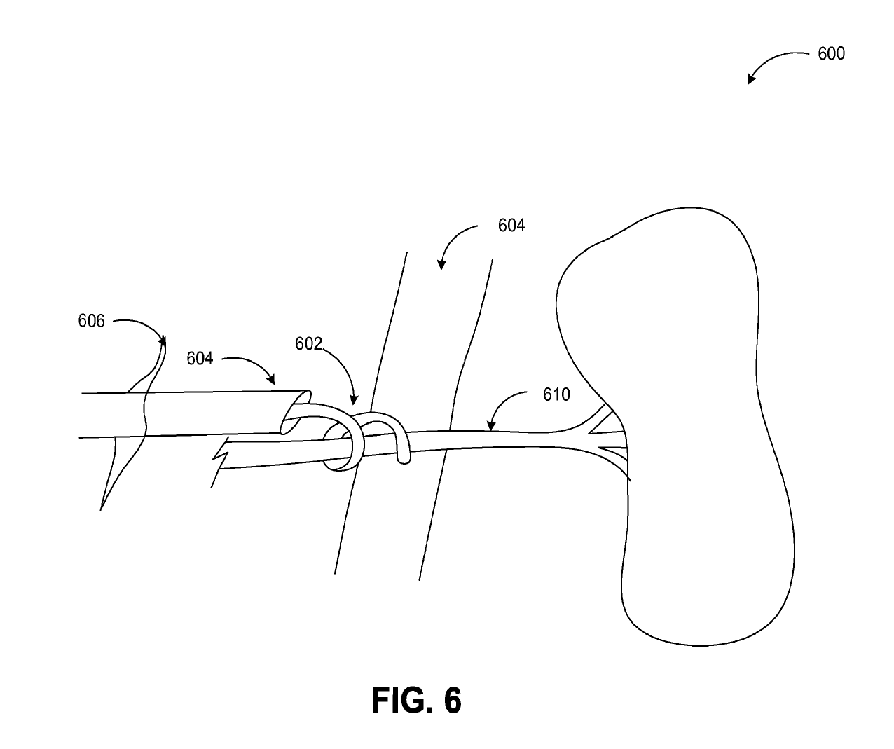

FIG. 6 illustrates a vascular reinforcement device being delivered via a delivery tool and deployed into a coiled position around the left renal vein, arranged in accordance with at least some embodiments as described herein. As illustrated in diagram 600, a coil 602 may be delivered and deployed into a coiled position around a left renal vein employing a laparoscopic procedure. The coil 602 may be deployed into the coiled position around the left renal vein 610 at a location where the left renal vein 610 crosses the aorta 604 for protecting the left renal vein from compression due to increased abdominal pressure.

A delivery tube 604 may provide percutaneous access to the abdominal cavity via at least one incision 606, and the coil 602 may be delivered through the delivery tube 604. The coil 602 may initially be configured in an uncoiled position such that the coil 602 is in a substantially long, straight and flat configuration for delivery into the abdomen through the delivery tube 604. The coil 602 may be guided through the delivery tube 604 for deployment into the abdomen employing a guiding tool. As the coil 602 emerges from the open distal end of the delivery tube 604, the coil 602 may automatically form into the coiled position around the left renal vein 610.

While embodiments have been discussed above using specific examples, components, and configurations, they are intended to provide a general guideline to be used for—providing a vascular reinforcement device for protecting the left renal vein from external pressures. These examples do not constitute a limitation on the embodiments, which may be implemented using other components, modules, and configurations using the principles described herein. Furthermore, actions discussed above may be performed in various orders, especially in an interlaced fashion.

According to some examples, the present disclosure describes a vascular reinforcement device for preventing compression of a blood vessel. The vascular reinforcement device may include a coil formed of a flexible material, the coil configured to wrap around a blood vessel for reducing an external pressure applied to the blood vessel. According to some examples, the coil may have a substantially circular cross-sectional shape. According to some examples, coil may have a diameter in a range configured to fit around the blood vessel. According to some examples, the coil may be configured to be unrolled into a straight and flat uncoiled position for delivery into an internal area of a body.

According to some examples, the flexible material may have a substantially rectangular cross-sectional shape when in the uncoiled position. The flexible material in the uncoiled position may have a width and length in a range for enabling the flexible material in the uncoiled position to be inserted within a surgical delivery tube.

According to some examples, the coil may wrap around a left renal vein at the location where an aorta crosses the left renal vein. The flexible material may be a thin polymer material. The polymer material includes one or more of polyamides, polyethylene, polypropylene, polyester, polyurethane, polystyrene, polysufone and/or polyethersulfone. The flexible material may be a bio-absorbable material. The bio-absorbable material may be selected from one or more of: include polyglycolic acid, polylactic acid, and polydioxanone. According to some examples, the flexible material may be a metal material. The metal may be selected from one of stainless steel or nickel titanium alloy.

According to some examples, the coil when wrapped in the coiled position around the blood vessel may be configured to resist an applied external pressure of about 50 mmHg to about 100 mmHg. The coil may be configured to be delivered in the uncoiled position and deployed into the coiled position around the blood vessel. The coil may be configured to be delivered in the uncoiled position employing a laparoscopic procedure. The coil may be configured to deploy into the coiled position around the blood vessel employing the laparoscopic procedure.

According to some examples, the present disclosure also describes a method of preventing compression of a vein from external bodily tissue forces. The method may include providing a coil formed of a flexible material, and wrapping the coil in around a blood vessel for reducing an external pressure applied to the blood vessel.

According to some examples, the method may include configuring the coil to have a substantially circular cross-sectional shape. The method may include configuring the coil to have a cross-sectional internal diameter in a range configured to fit around the blood vessel. The method may include configuring the flexible material to be unrolled into a straight and flat uncoiled position for delivery into an internal area of a body. The method may include configuring the flexible material to have a substantially rectangular cross-sectional shape when in the uncoiled position.

According to some examples, the method may include configuring the flexible material in the uncoiled position to have a width and length in a range for enabling the flexible material in the uncoiled position to be inserted within a surgical delivery tube. The method may include positioning the coil in the coiled position around a left renal vein at the location where the left renal vein crosses an aorta. The method may include composing the flexible material from a thin polymer material.

According to some examples, the polymer material includes one or more of polyamides, polyethylene, polypropylene, polyester, polyurethane, polystyrene, polysufone and/or polyethersulfone. According to some examples, the method may include composing the flexible material from a bio-absorbable material selected from one or more of polyglycolic acid, polylactic acid, and polydioxanone. The method may include composing the flexible material from a metal material. The metal may be selected from one of stainless steel or nickel titanium alloy.

According to some examples, the method may include configuring the coil when wrapped around the blood vessel to resist an applied external pressure of about 50 mmHg to about 100 mmHg. The method may include delivering the coil in the uncoiled position employing a laparoscopic procedure. The method may include deploying the coil into the coiled position around the blood vessel employing the laparoscopic procedure.

According to other examples, the present disclosure also describes a system for preventing compression of a vein from external bodily tissue forces. The system may include a surgical delivery tube for providing percutaneous access to an internal area of a body via at least one incision and a vascular reinforcement device including a coil formed of a flexible material, the coil configured to wrap around a blood vessel for reducing an external pressure applied to the blood vessel, wherein the coil may be configured in an initial uncoiled position for delivery to the internal area of the body via the delivery tube and deployed into the coiled position around the blood vessel.

According to some examples, the internal area of the body may be a location in the abdominal cavity near the aorta. The initial uncoiled position of the vascular reinforcement device may include the coil unrolled into a straight and flat uncoiled position for fitting within the delivery tube. The coil in the uncoiled position may be guided into the internal area of the body employing a guiding tool. The coil may be configured to coil around the blood vessel as the coil emerges from the delivery tube.

According to some examples, the coil may be deployed into the coiled position over a left renal vein at a location where the left renal vein crosses the aorta. The flexible material may be a thin polymer material. The polymer material may be one of polyamides, polyethylene, polypropylene, polyester, polyurethane, polystyrene, polysufone and/or polyethersulfone. The flexible material may be a bio-absorbable material. The flexible material may be a metal material. The metal may be nickel titanium alloy.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A vascular reinforcement device for preventing compression of a blood vessel, comprising:
a thin bio-absorbable polymeric material including one or more of: polyamides, polyethylene, polypropylene, polyester, polyurethane, polystyrene, polysufone and polyethersulfone; and
a coil existing in a coiled position or uncoiled position, wherein
when in the coiled position, the coil has a substantially circular cross-sectional shape with a diameter in a range configured to wrap around a blood vessel to resist an external pressure applied to the blood vessel; and
when in the uncoiled position, the coil has a substantially rectangular cross-sectional shape with a width and length in a range for enabling the thin polymeric material to be inserted within a surgical delivery tube and into an internal area of the body.

2-6. (canceled)

7. The vascular reinforcement device of claim 1, wherein the coil wraps around a left renal vein at the location where an aorta crosses the left renal vein.

8-10. (canceled)

11. The vascular reinforcement device of claim 1, wherein the bio-absorbable material is selected from one or more of: include polyglycolic acid, polylactic acid, and polydioxanone.

12. The vascular reinforcement device of claim 1, wherein the flexible material is a metal material.

13. The vascular reinforcement device of claim 12, wherein the metal is selected from one of stainless steel or nickel titanium alloy.

14-15. (canceled)

16. The vascular reinforcement device of claim 1, wherein the coil is configured to be delivered in the uncoiled position employing a laparoscopic procedure.

17. The vascular reinforcement device of claim 16, wherein the coil is configured to deploy into the coiled position around the blood vessel employing the laparoscopic procedure.

18. A method of preventing compression of a vein from external bodily tissue forces, comprising:
providing a coil in the coiled position having a substantially circular cross-sectional shape with an internal diameter in a range configured to fit around a blood vessel formed of a flexible material allowing for the reduction of an external pressure applied to the blood vessel; and
configuring the flexible material to be uncoiled into a straight and flat uncoiled position with a substantially rectangular cross-sectional shape with a width and length in a range to enable the flexible material to be inserted within a surgical delivery tube for delivery into an internal area of a body.

19-23. (canceled)

24. The method of claim 18, further comprising:
positioning the coil in the coiled position around a left renal vein at the location where the left renal vein crosses an aorta.

25. (canceled)

26. The method of claim 18, wherein the polymer material includes one or more of polyamides, polyethylene, polypropylene, polyester, polyurethane, polystyrene, polysufone and/or polyethersulfone.

27. The method of claim 18, further comprising:
composing the flexible material from a bio-absorbable material selected from one or more of polyglycolic acid, polylactic acid, and polydioxanone.

28. The method of claim 18, further comprising:
composing the flexible material from a metal material.

29. The method of claim 28, wherein the metal is selected from one of stainless steel or nickel titanium alloy.

30. The method of claim 18, further comprising:
configuring the coil when wrapped around the blood vessel to resist an applied external pressure of about 50 mmHg to about 100 mmHg.

31. The method of claim 18, further comprising:
delivering the coil in the uncoiled position employing a laparoscopic procedure.

32. The method of claim 30, further comprising:
deploying the coil into the coiled position around the blood vessel employing the laparoscopic procedure.

33. A system for preventing compression of a vein from external bodily tissue forces, comprising:
a surgical delivery tube for providing percutaneous access to an internal area of a body via at least one incision;
a vascular reinforcement device including a coil formed of a flexible polymeric material, wherein the flexible material is a bio-absorbable material selected from one or more of polyglycolic acid, polylactic acid, and polydioxanone, the coil configured to wrap around a blood vessel to reduce for an external pressure applied to the blood vessel, wherein the coil is configured in an initial straight and flat uncoiled position for delivery to the internal area of the body via the delivery tube and deployed into the coiled position around the blood vessel as the coil emerges from the delivery tube.

34. The system of claim 33, wherein the internal area of the body is a location in the abdominal cavity near the aorta.

35. (canceled)

36. The system of claim 33, wherein the coil in the uncoiled position is guided into the internal area of the body employing a guiding tool.

37. (canceled)

38. The system of claim 33, wherein the coil is deployed into the coiled position over a left renal vein at a location where the left renal vein crosses the aorta.

39. (canceled)

40. The system of claim 33, wherein the polymer material is one of polyamides, polyethylene, polypropylene, polyester, polyurethane, polystyrene, polysufone and/or polyethersulfone.

41. (canceled)

42. The system of claim 33, wherein the flexible material is a metal material.

43. The system of claim 42, wherein the metal is nickel titanium alloy.
